(19) United States
(12) Patent Application Publication
(10) Pub. No.: US 2025/0177695 A1
(43) Pub. Date: Jun. 5, 2025

(54) Customized Urinary Catheters and Methods for Making

(71) Applicant: Hollister Incorporated, Libertyville, IL (US)

(72) Inventors: Michael G. Murray, Ballina (IE); David J. Farrell, Ballina (IE); Vincent Naughton, Sligo (IE); Kevin Murnaghan, Ballina (IE)

(21) Appl. No.: 18/839,176

(22) PCT Filed: Feb. 16, 2023
(86) PCT No.: PCT/US23/62746

§ 371 Date: Aug. 16, 2024

Related U.S. Application Data
(60) Provisional application No. 63/311,297, filed on Feb. 17, 2022.

(51) Int. Cl. A61M 25/00; B33Y 50/00; B33Y 80/00

(52) U.S. Cl. CPC A61M 25/0069; A61M 25/001; A61M 25/0023; A61M 25/007; B33Y 50/00; B33Y 80/00; A61M 2205/60; A61M 2207/00

(57) ABSTRACT

A method of making a customized catheter for an end user, comprising: capturing images of an end user's urethra; creating a 3D image of the end user's urethra; identifying areas of interest of the end user's urethra; generating a software or computerized model of a customized catheter using the 3D image, wherein the software or computerized model is modified based on the areas of interest; and 3D printing at least a portion of the customized catheter from the model and catheter made by this method

### CROSS-REFERENCE TO RELATED APPLICATION

This application claims the benefit of U.S. Provisional Application Ser. No. 63/311,297, filed Feb. 17, 2022, the disclosure of which is hereby incorporated by reference in its entirety.

### TECHNICAL FIELD

The present disclosure generally relates to urinary catheters that are customized for a particular user and methods of making the same, and more particularly, to urinary catheters that are parametrically designed for a user and manufactured using additive manufacturing or 3D printing.

### BACKGROUND

Intermittent urinary catheters are commonly used by those who suffer neurogenic conditions that affect bladder function, such as but not limited to SCI or multiple sclerosis, and for those with urinary retention. Urinary catheters drain urine from the bladder through a shaft and out a distal end.

Catheters are typically produced by a manufacturing process with a set of manufacturing rules. For a manufacturing process, such as extrusion, the catheters must be of a uniform shape and thickness and be made of the same material, such that the catheters can be mass produced without stopping or changing the process. The catheters produced by these traditional processes are identical and although useful for the typical consumer, may not work as well for someone with an abnormally shaped urethra that may include irregularities.

Some catheter users may have abnormally shaped urethras. For instance, the urethra may include irregularities such as curves, constrictions, and kinking. As described above, normal manufacturing processes cannot account for these irregularities, making the traditional catheter less than ideal for those patients with these irregularities.

There remains a need for catheters which can be utilized for users with urethras that include irregularities.

### SUMMARY

There are several aspects of the present subject matter which may be embodied separately or together in the devices and systems described and claimed below. These aspects may be employed alone or in combination with other aspects of the subject matter described herein, and the description of these aspects together is not intended to preclude the use of these aspects separately or the claiming of such aspects separately or in different combinations as set forth in the claims appended hereto.

In one aspect, a method of making a customized catheter for an end user includes capturing images of the end user's urethra, creating a 3D image of the end user's urethra, identifying areas of interest of the urethra, generating a software or computerized model of a customized catheter using the 3D image, and 3D printing the customized catheter from the model. The model is modified based on the identified areas of interest.

In another aspect, a catheter includes a tube with a proximal end and a distal end, and a 3D printed tip attached to the proximal end of the tube.

In another aspect, a catheter includes a tube with a proximal end and a distal end and an internal lumen. The catheter tube has a first cross-sectional width of the internal lumen at a first location and a second cross-sectional width of the internal lumen at a second location along the tube. The first cross-sectional width is different than the second cross-sectional width.

### BRIEF DESCRIPTION OF DRAWINGS

FIG. 1 is a perspective view showing a videoscope and method according to one embodiment of the current disclosure.

FIG. 2A is a first perspective view of a first patient's urethra.

FIG. 2B is a second perspective view of the first patient's urethra in FIG. 2A.

FIG. 2C is a third perspective view of the first patient's urethra in FIG. 2A.

FIG. 3A is a first perspective view of a second patient's urethra.

FIG. 3B is a second perspective view of the second patient's urethra in FIG. 3A.

FIG. 3C is a third perspective view of the second patient's urethra in FIG. 3A.

FIG. 4 is a perspective view of a 3D image of the first patient's urethra.

FIG. 5 is a perspective view of a 3D image of the second patient's urethra.

FIG. 6 is a flow chart of the steps taken to produce a custom catheter for a user.

FIG. 7. is a top view of a prior art catheter.

FIG. 8 is a top view of a catheter tip and a catheter tube of the current disclosure.

FIG. 9 is a top view of a catheter tip according to one embodiment of the current disclosure.

FIG. 10 is a top view of a catheter tip according to one embodiment of the current disclosure.

FIG. 11 is an enlarged section of the catheter tip of FIG. 10A.

FIG. 11A are enlarged cross-sections showing an alternative of the open cell or pore configuration of the porous catheter tip.

FIG. 11B are enlarged cross-sections showing another alternative of the open cell or pore configuration of the porous catheter tip.

FIG. 11C are enlarged cross-sections showing another alternative of the open cell or pore configuration of the porous catheter tip.

FIG. 11D are enlarged cross-sections showing another alternative of the open cell or pore configuration of the porous catheter tip.

FIG. 12 is a top view of a catheter tip according to one embodiment of the current disclosure.

FIG. 13 is a top view of a prior art catheter.

FIG. 14 is a cross section of the catheter of FIG. 13.

FIG. 15A is an enlarged section of the catheter of FIG. 13.

FIG. 15B is an enlarged section of the catheter of FIG. 13.

FIG. 16 is a top view of a catheter according to one embodiment of the current disclosure.

FIG. 17 is a top view of a catheter according to one embodiment of the current disclosure.

### DETAILED DESCRIPTION OF THE ILLUSTRATED EMBODIMENTS

The embodiments disclosed herein are for the purpose of providing a description of the present subject matter, and it is understood that the subject matter may be embodied in various other forms and combinations not shown in detail. Therefore, specific embodiments and features disclosed herein are not to be interpreted as limiting the subject matter as defined in the accompanying claims.

The present disclosure is directed to custom catheters that are parametrically designed for a particular user and methods of making the same. Images of the user's urethra are captured and used to create a 3D image of the urethra. Areas of interest, such as abnormal areas or irregularities of the urethra are identified and a model of an appropriate catheter for the user is generated. The model can then be used to 3D print at least a portion of the customized catheter or 3D print the entire catheter, taking into account the areas of interest so that the catheter is custom to the user. In one alternative, the model can be used to 3D print at least a portion of the catheter shaft or the entire catheter shaft. The present disclosure is also directed to 3D printing select portions of a catheter, such as the tip portion, for combination with a traditionally manufactured catheter portion.

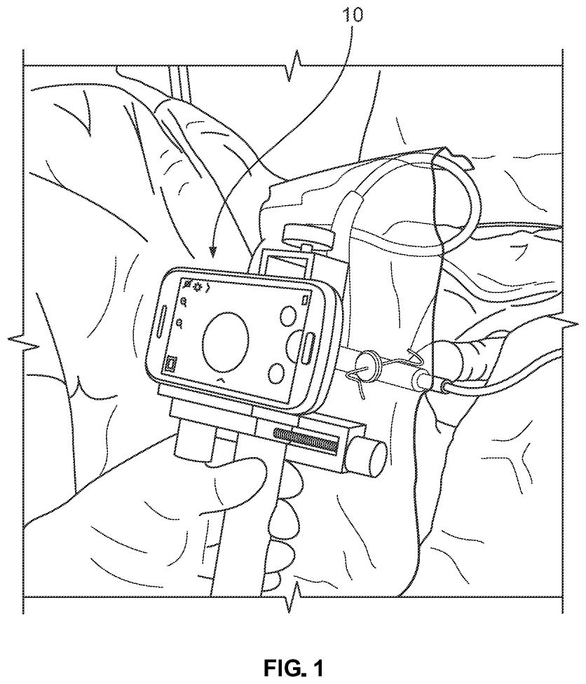

FIG. 1 illustrates a first step in the process of creating a custom catheter for an end user. The urethra is imaged. This imaging can be done in a number of ways and can include imaging machines such as X-rays, MRI, CT (computer tomography scans), Ultrasound, and Ultrasonography, including any suitable technology for imaging a person's body from the outside. The imaging can also be performed internally with a device such as a videoscope or any device capable of viewing the surface from inside the urethra's lumen. FIG. 1 includes an embodiment of the current disclosure in which a videoscope 10 is inserted into the patient's (end user's) urethra and produces images of the internal structure and/or surface.

Along with imaging and/or measuring the inner surface of the urethra, the inner topology of the urethra may be analyzed by observing or calculating the force required to insert a catheter along different portions of the urethra. Additional force may be required when the urethra is constricted or otherwise abnormally shaped. This force can be measured by any known pressure device. An internal imaging device, such as a scope, can include a pressure device. The pressure device can be a separate instrument, or a person can qualitatively measure the force required at any given point along the urethra and compare it to previous points. These force measurements, however taken, can be used in determining the shape of a user's urethra and ultimately one or more of the shaped, contour, rigidity, and pliability of the final customized catheter for a user.

Images may be taken of a given end user's urethra at any number of different points or angles along the urethra so that the internal pathway of the urethra may be accurately mapped. When sufficient images or measurements of the urethra have been taken, composite 3D images of the urethra can be mapped for plotting an image of the entire length of the urethra. For example, the 3D image may be created using photogrammetry.

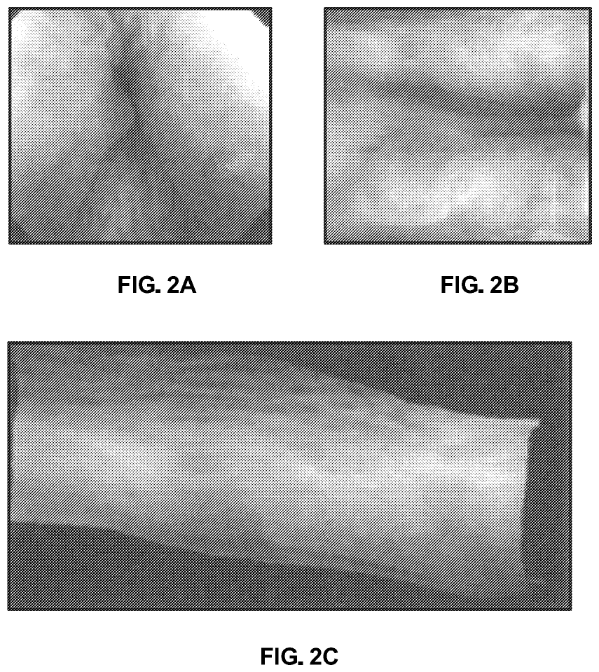

FIGS. 2A-2C are examples of urethra images from a first catheter user, showing different points or angles along the urethra. Although three images are shown, more or less images may be taken of a given user's urethra at any number of different points or angles along the urethra so that the internal pathway of the urethra may be accurately mapped.

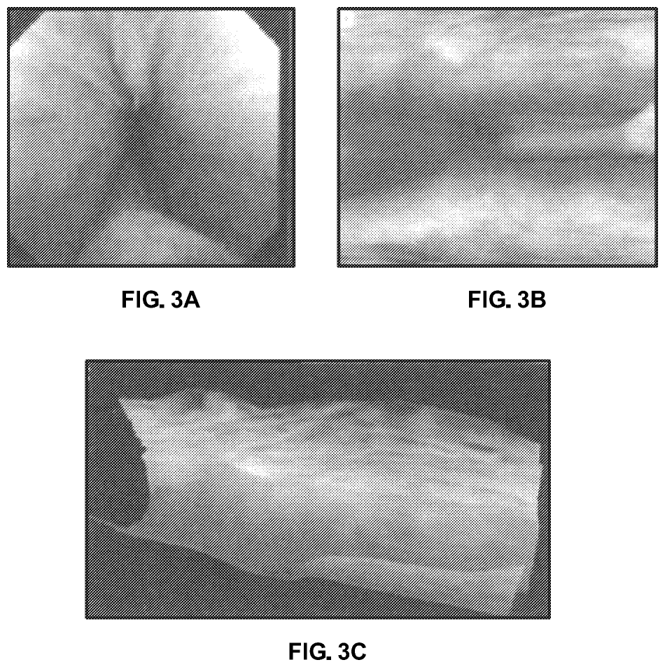

FIGS. 3A-3C are examples of urethra images from a second user, showing different points or angles along the urethra. Although three images are shown, more or less images may be taken of a given user's urethra at any number of different points or angles along the urethra so that the internal pathway of the urethra may be accurately mapped.

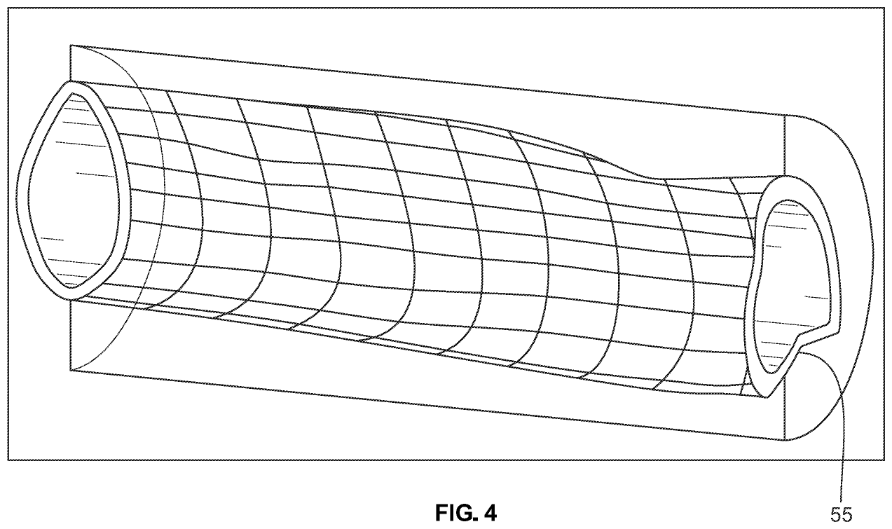

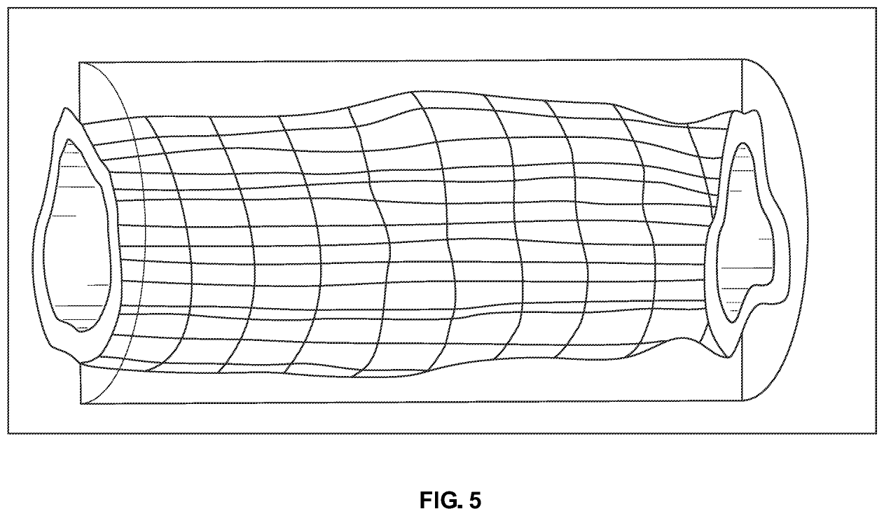

FIGS. 4 and 5 show resulting 3D composite images for the first user and second user, respectively. The 3D composite image can then be used to identify abnormal (irregular) areas of the urethra. This is done by an algorithm or other computer function that can be used to pinpoint areas of physiology of interest. These abnormal or irregular areas may include irregularities such as curves, constrictions, urethral strictures, stenosis of the urethra, false passages, enlarged prostrates, and kinking. For example, a stricture 55, which is a narrowing of the urethra that can be due to scarring, is shown in FIG. 4.

Afterward, a software or computer model of a catheter customized to an end user's urethra is created. Software, such as parametric mesh generation software builds the model, such as a mesh. Optionally, before it is sent to print, the software checks to ensure that features such as the right size, lengths, tips, stiffnesses, eyelets etc. are correct and ready for printing.

At any point along the process a catheter model and the resulting catheter may be assigned or labeled with a unique identifier or identification that can follow along with the process. Ideally at the imaging step a unique identifier is assigned, but the identifier can be added at any point in the process to associate an end user with the end product catheter and model, and reduce or eliminate any errors in the associated end user with the correct end product catheter and model. The model may be saved for future use and retrieval.

The model or mesh is then sent to be manufactured by 3D printing or additive manufacturing. Such 3D printing devices may include FDM Fused Deposition Modelling and Multi Jet Fusion. The catheter or catheter components are printed. If multiple components are produced, then they are joined to create the catheter and packaged appropriately. The packaging may be specific to the end user and include a unique identifier or an end user's name to distinguish the end user on the packaging. The packaging color and/or design may be customized or personalized to the end user. In future manufacturing, the saved software or computerized model may be retrieved and customized catheters may be 3D printed from the saved software or computerized model.

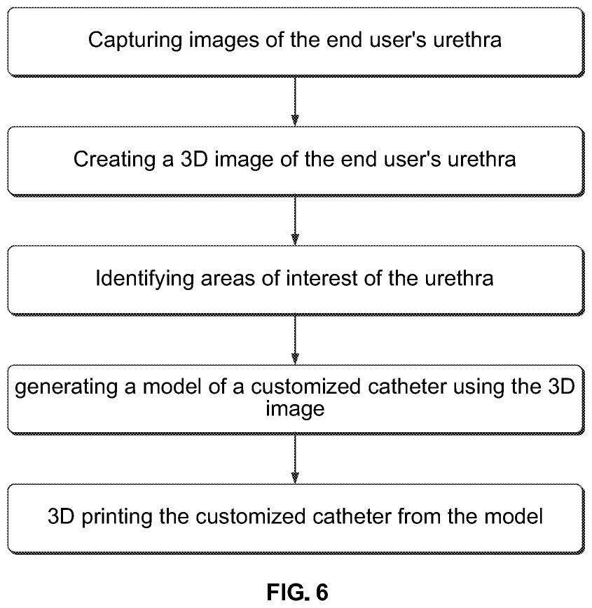

FIG. 6 includes a flow chart for the process of creating a custom catheter of the current disclosure. The process, as described above, includes capturing images of the end user's urethra, creating a 3D image of the end user's urethra, identifying areas of interest (such as abnormal/irregular areas) of the urethra, generating a software or computerized model of a customized catheter using the 3D image, and 3D printing the customized catheter from the model. The model is modified based on the identified abnormal areas.

While it is possible to 3D print the entire catheter, it is also possible to 3D print a smaller portion of the catheter, such as the tip portion or tube portion and then connect the 3D printed portion to the additional portions of the catheter.

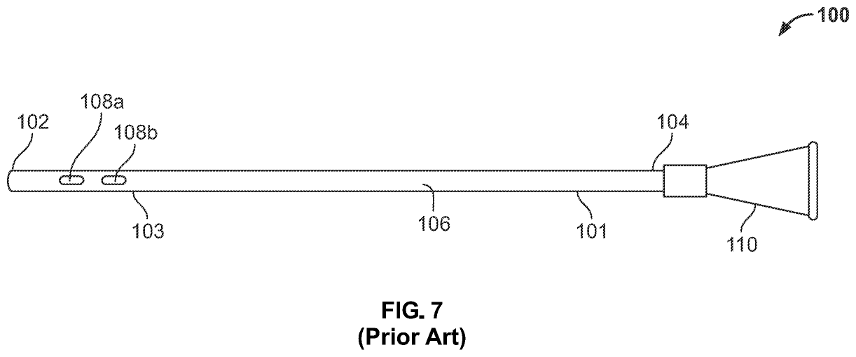

FIG. 7 illustrates a prior art catheter. The urinary catheter 100 has a catheter shaft 106 with a proximal portion 103 which has a proximal end 102 and a distal portion 101 which has a distal end 104. The catheter and/or catheter shaft 106 may be made of a polymeric material. The polymeric material may include, but is not limited to, polyethylenes, polypropylenes, polyvinylchlorides, polytetrafluoroethylenes, polyvinylacetates, polystyrenes, polyesters, polyurethanes, polyamides, ethylene vinyl acetate copolymers, and combinations thereof.

The proximal end 102 of the catheter shaft has a tip that can be any appropriate shape for insertion through the urethra and into the bladder. In one embodiment, the proximal end 102 has a straight rounded shape, such as the illustrated nelaton shape. Other shapes are applicable such as curved and tapered (Coudé, Tiemann, Olive tip).

The proximal portion 103 of the catheter shaft 106 may include one or more draining holes or eyelets 108a, 108b in communication with an inner lumen (not shown) of the proximal portion 103 of the catheter shaft.

As shown in the figure, the eyelets 108a, 108b can be distally spaced from the proximal tip of the proximal end of the catheter. However, the eyelet or eyelets 108a, 108b may be closer to the proximal tip or may arranged in any effective manner. In one alternative, the eyelets 108a, 108b may be in or at the proximal end 102. The eyelet(s) 108a, 108b may be configured to be located within a bladder when the catheter is inserted to drain urine from the bladder.

The distal end portion 104 may include a funnel 110 or other draining aid. The distal end may include a connector for connecting to a drainage bag.

The catheter of FIG. 7, with or without any minor modifications, can be produced by 3-D printing or additive manufacturing as a whole. Different sections, or portions, of the catheter may be comprised of the same or different material or mixture of materials. Another option can be to manufacture portions separately and then join them together to form the final catheter end product, such as 3D printing the tip portion and injection molding the tube or body portion of the catheter or vice versa.

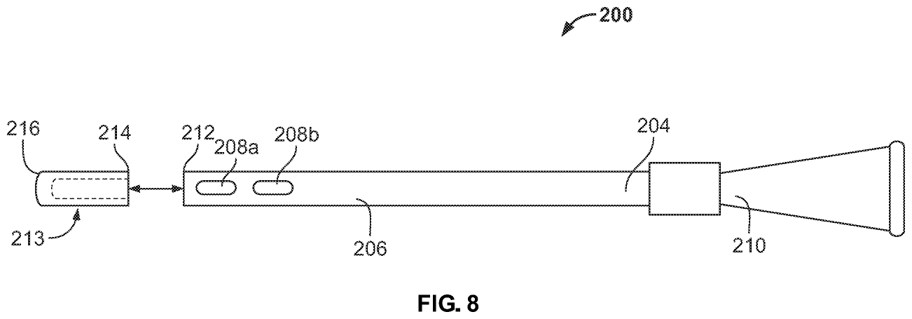

As illustrated in FIG. 8, the catheter 200 may include two separate portions, a tip portion 213 and a catheter body portion or catheter tube 206. The catheter may include similar features as outlined in FIG. 7, and these features are numbered similarly. However, the tip portion is constructed or manufactured separately. The tip portion 213 includes a proximal end 216 and a distal end 214 for joining to the catheter body. Although a straight or nelaton tip is shown, any shaped catheter tip may be used. The catheter tube 206 includes a distal end 204 and a proximal end 212 for joining to the tip portion 213.

One or both pieces may be produced by 3D printing or additive manufacturing. One or both pieces may also be produced by a different manufacturing process, such as injection molding or extrusion. The tip portion and the body portion may then be combined. This combination can be done in any of known means in the art, such as welding, bonding or heat sealing. The two portions may be separately 3D printed so that the pieces complement each other, or can be easily inserted into each other. For instance, the tip may be hollow or partially hollow so that a portion of the body portion may be inserted into the tip portion. In other alternatives, the tip can be solid or non-hollow. This joining can be the sole method of joining the two portions or can be used in conjunction with another conventional method of joining two components of a device, like welding, bonding or heat sealing. For example, the tip and the tube may be attached at a joint line. In one alternative, the portions are joined so that there is a smooth transition on the outer surface of the catheter where the two portions are joined together.

As shown in FIG. 9, the tip 313 may be curved or a flex coudé tip. Any tip shape can be utilized, especially when 3D printing or using additive manufacturing to manufacture the tip.

The tip and catheter body may be formed of the same or different compositions. The tip and/or the catheter tube's body can be formed of the same or different combination of polymeric material. The polymeric material may include, but is not limited to, polyethylenes, polypropylenes, polyvinylchlorides, polytetrafluoroethylenes, polyvinylacetates, polystyrenes, polyesters, polyurethanes, polyamides, ethylene vinyl acetate copolymers, and combinations thereof.

The tip of the catheter can be softer material than the tube of the catheter, which aids in easier catheter insertion, control and manipulation. Alternatively, the tube of the catheter may be softer than the tip of the catheter. The varying in flexibility may be especially beneficial in a male catheter where the catheter is inserted along the curved or serpentine path of the urethra.

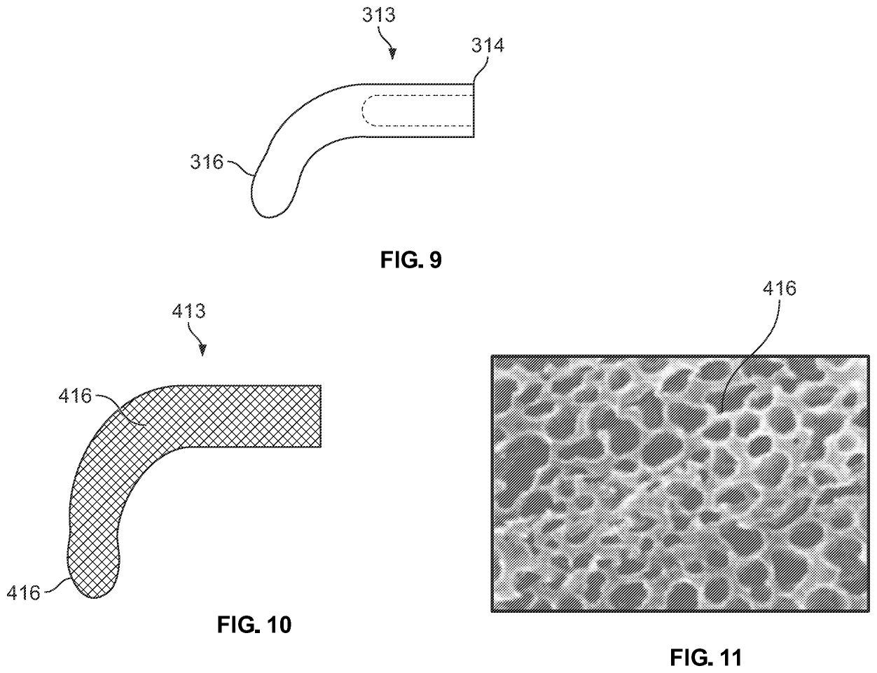

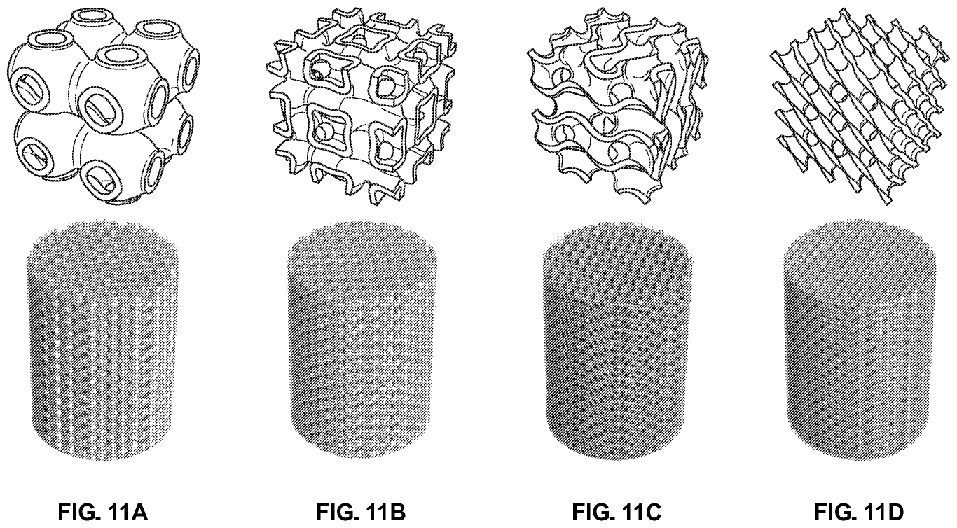

The 3D printed tip can have other unique properties. In FIG. 10 a porous curved or flex olive tip is shown. FIG. 11 is an exploded view of the tip 413 with pores 416. Although the tip is shown as being comprised of a single porous material, the tip may include a mixture of material with varying porosity. The porous material may allow drainage of urine from the bladder to pass into the internal drainage lumen (not shown) of the catheter tube. The porous material can include any common type of porous material such as foam, sponge, sintered, metal, or ceramic. The porous material may be a 3D printed lattice. Different lattice geometries may be used such as honeycomb, truss, shone gyroic, etc. The porous material may have an open or interconnected pore structure or configuration, wherein the voids of the pores are in communication with voids of other pores. FIGS. 11a, 11b, 11c, and 11d illustrate examples of porous open cell or pore structures. FIG. 11a shows circular pores. FIG. 11b shows square shaped pores. FIG. 11c shows pores in waved channels. FIG. 11d shows pores in diagonal channels. The pores of the porous material may vary in average pore size, range of pore size, average pore density, number of pores, pore shape, and shape and size of pore passageways (varying in internal surface features). In one embodiment, the pore size may be between 0.25 mm and 1 mm in diameter. The pores may be in a random or parametric configuration. The pores could occupy a pore to substrate ratio of anywhere between about 1:4 and about 2:1. These may vary based on desired qualities of the material. For instance, lower pore ratios may decrease micro trauma.

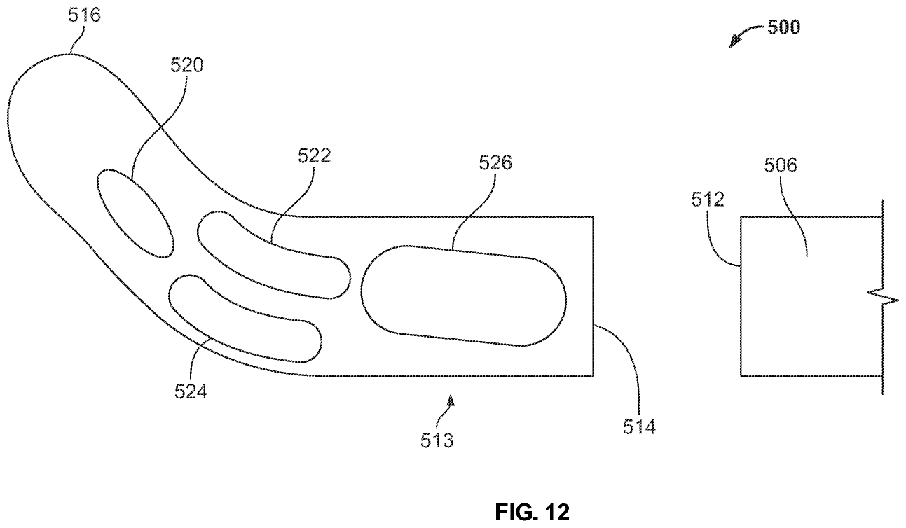

FIG. 12 shows an additional embodiment of the current invention. Tip portion 513 may include at least one eyelet or opening. As shown in FIG. 12, the tip portion 513 may include a plurality of eyelets, 520, 522, 524, and 526. The eyelets may be uniform in shape or eyelets may be uniquely or irregularly shaped. For example, eyelets shapes include geometric shapes, round/oval, geometric shapes with rounded corners, hourglass shapes, amoeba shaped, knife shaped, boomeranged shaped, etc. Eyelets may be different from at least one other eyelet in at least one of size, shape, and position. In one alternative each eyelet is different from the other eyelets. Although four eyelets are shown in FIG. 12, it is within the scope of this disclosure to include one, two, three, or more than four eyelets. The eyelets may be arranged in a pattern or randomly spaced. The eyelets, as shown in the previous figures, may also be on the catheter body or tube. Additionally, eyelets may also be present on both the tip and tube or body portion of the catheter.

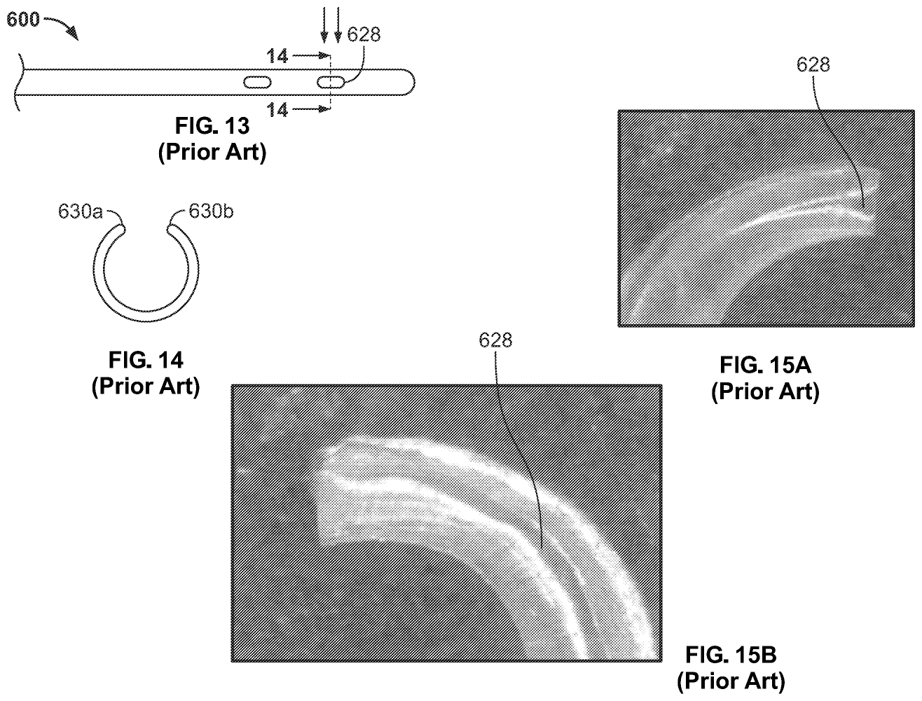

Utilizing 3D printing for the tip of the catheter offers not only variety in shape, sizes, positions and numbers of eyelets present on any given catheter, but also allows for producing a catheter eyelet that is not only smooth and rounded on the outside but smooth and rounded on the inside. For example, in the 3D printed tips of the present disclosure, the transition between the outer surface of the catheter tube and the surface of the wall defining the opening of the eyelet may be a rounded or smooth transition. Furthermore, there may also be a rounded or smooth transition between the inner surface of the catheter tube and the catheter tube wall defining the opening of the eyelet in communication with the lumen of the catheter tube. Catheter eyelets that are produced by typical manufacturing processes such as cold punch, heated punch or ultrasonic means can cause a sharp catheter eyelet, shown in FIGS. 13, 14, 15A and 15B (Prior Art). The eyelet 628 of catheter 600 can have sharp edges 630a and 630b. During the catheterization process, the tissue from the urethra can get sucked into these sharp catheter eyes. By allowing for the smooth rounded inside and outside, 3D printing can prevent this discomfort to the user.

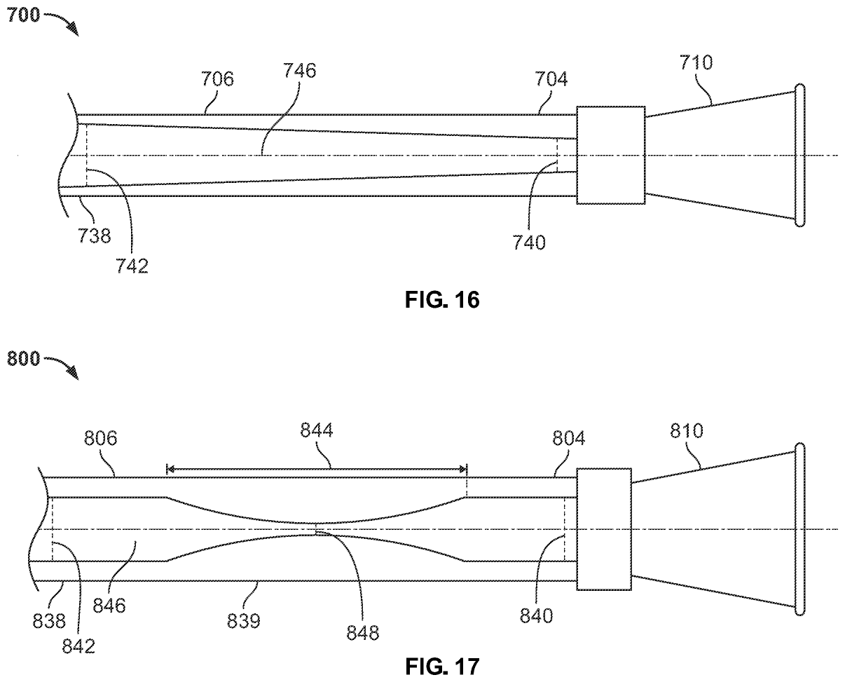

The geometry of portions of the catheter tube are easily modified and changed by utilizing 3D printing. The cross-sectional width of the internal lumen 746 of the catheter 700 may vary. This may be done by increasing and/or decreasing the width or thickness of the catheter tube wall along the length of the tube. FIG. 16 shows a first embodiment of varying internal lumen cross-sectional width. A first internal lumen cross-sectional width 742 near a proximal portion 738 can be larger than a second internal lumen cross-sectional width 740 at a distal end 704 of the catheter tube. The walls of the catheter tube may be thicker at the distal end 704 and thinner at the proximal end portion 738. In FIG. 17, another embodiment of varying catheter wall thickness or internal lumen cross-sectional width is shown. The catheter has a first internal lumen cross-sectional width 842 at a proximal portion 838 and a second internal lumen cross-sectional width 840 at a distal end of the catheter. The first and second internal lumen cross-sectional width may be identical. A middle portion 844 of the catheter 800 may have a thicker wall and a smaller internal lumen cross-sectional width 848 at a third point 839 along the length of the catheter 800.

The catheter portions included in the current disclosure, that are not formed by 3D printing, may be formed by any known manufacturing process. The catheter portions may be formed in a single process or multiple processing steps. The catheter portions may be formed by different types of molding, more particularly by injection molding. The catheter portions may also be formed by heating or extruding a portion or portions to the desired shape.

The catheters included in the current disclosure may be, but not limited to, hydrophilic catheters and parts of the catheter shaft may include a hydrophilic coating. When the hydrophilic coating is wetted or hydrated with a hydration medium, such as water, it becomes lubricious which eases introduction of the device into the body and aids in reducing pain and discomfort associated with such introduction. The hydrophilic coating can be a single layer or multilayer hydrophilic coating. Multiple layered coating can include at least a base coat and top layer. The catheters may also include a gel on the outer surface to aid with insertion.

The catheters included in the current disclosure, optionally, also may include a thin flexible sleeve that covers at least a section of the outer surface of the catheter shaft. The sleeve may be formed of any variety of thin flexible polymeric film materials, such as polyethylene, plasticized PVC, polypropylene, polyurethane or elastomeric hydrogels. When the catheter includes a hydrophilic coating thereon, the sleeve may be liquid and/or vapor permeable so as to allow liquid and/or vapor therethrough to hydrate the hydrophilic coating while the catheter is stored within a package. Alternatively, the sleeve may include a hydration liquid or a foamed hydration liquid within the sleeve and in contact with the hydrophilic material.

Any of the above-described catheters may be used by male or female patients. Male catheters tend to differ from female catheters in length because of difference in urethra length. Female catheters of the current disclosure may be shorter overall and may also have a shorter distal end portion.

The catheters of the present disclosure may be sterilized prior to use. The catheters may be sterilized by applying a sufficient amount of radiation, such as (but not limited to) gamma or E-Beam radiation. The catheters can be sterilized with radiation while the hydrophilic coating is in contact with the wetting fluid.

It will be understood that the embodiments described above are illustrative of some of the applications of the principles of the present subject matter. Numerous modifications may be made by those skilled in the art without departing from the spirit and scope of the claimed subject matter, including those combinations of features that are individually disclosed or claimed herein. For these reasons, the scope hereof is not limited to the above description but is as set forth in the following claims, and it is understood that claims may be directed to the features hereof, including as combinations of features that are individually disclosed or claimed herein.

1. A method of making a customized catheter for an end user, comprising:
capturing images of an end user's urethra;
creating a 3D image of the end user's urethra;
identifying areas of interest of the end user's urethra;
generating a software or computerized model of a customized catheter using the 3D image, wherein the software or computerized model is modified based on the areas of interest; and
3D printing at least a portion of the customized catheter from the model.

2. The method of claim 1, further comprising labeling the customized catheter with a unique identification for the end user.

3. The method of any one of claim 1, further comprising saving the software or computerized model.

4. The method of claim 3, further comprising assigning saved software or computerized model with a unique identification for the end user.

5. The method of claim 3, further retrieving the saved software or computerized model and 3D printing additional customized catheters from the saved software or computerized model.

6. The method of claim 1, wherein the capturing is performed by a videoscope.

7. The method of claim 1, wherein the creating a 3D image comprises using photogrammetry.

8. The method of claim 1, wherein the areas of interest include irregular areas.

9. The method of claim 1, wherein the areas of interest include at least one from the group consisting of curves, constrictions, and kinking.

10. (canceled)

11. A catheter comprising:
a tube with a proximal end and a distal end; and
a 3D printed tip attached to the proximal end of the tube.

12. The catheter of claim 11, wherein the 3D printed tip includes a plurality of openings.

13. The catheter of claim 12, wherein the plurality of openings comprises irregular shaped openings.

14. The catheter of claim 12, wherein each opening is different from at least one other opening in at least one of size, shape, and position.

15. The catheter of claim 12, wherein at least one shape is oval.

16. The catheter of claim 11, wherein the 3D printed tip is at least partially hollow.

17. The catheter of claim 11, wherein the 3D printed tip is solid.

18. The catheter of claim 11, wherein a portion of the tube is inserted into the 3D printed tip.

19. The catheter of claim 11, wherein the 3D printed tip is attached to the tube at a joint line.

20. The catheter of claim 11, wherein the 3D printed tip is curved.

21. (canceled)

22. A catheter comprising:
a tube with a proximal end and a distal end and an internal lumen; and
a first cross-sectional width of the internal lumen at a first location and a second cross-sectional width of the internal lumen at a second location along the tube;
wherein the first cross-sectional width is different than the second cross-sectional width.

23.-28. (canceled)
